SEQUENCE LISTING (1 sequence)

SEQ ID NO: 1 - protein, 18 aa, synthetic construct
  REGION 1..18: note = A18 paptide
DWLKAFYDKVAEKLKEAF

(19) United States
(12) Patent Application Publication
(10) Pub. No.: US 2024/0410894 A1
(43) Pub. Date: Dec. 12, 2024

(54) ARTIFICIAL BIOMEMBRANE CONTAINING MEMBRANE PROTEIN

(71) Applicant: NATIONAL UNIVERSITY CORPORATION KOBE UNIVERSITY, Hyogo (JP)

(72) Inventors: Kenichi MORIGAKI, Hyogo (JP); Fumio HAYASHI, Hyogo (JP)

(73) Assignee: NATIONAL UNIVERSITY CORPORATION KOBE UNIVERSITY, Hyogo (JP)

(21) Appl. No.: 18/702,815

(22) PCT Filed: Oct. 14, 2022
(86) PCT No.: PCT/JP2022/038358

§ 371 Date: Apr. 19, 2024

(30) Foreign Application Priority Data
Oct. 22, 2021 (JP) 2021-173293

(51) Int. Cl. G01N 33/68; C07K 14/705; G01N 33/92

(52) U.S. Cl. CPC G01N 33/68; C07K 14/705; G01N 33/92

(57) ABSTRACT

Provided is an artificial biological membrane containing a membrane protein retaining lateral diffusivity. Also provided is a method of producing an artificial biological membrane containing a membrane protein retaining lateral diffusivity. The artificial biological membrane is obtained by a method of producing an artificial biological membrane, the method including a step of partially laminating a polymer lipid membrane (b) on the surface of a substrate (a), followed by the lamination of a fluid lipid membrane (c) on the surface of the substrate (a), on which the polymer lipid membrane (b) is not laminated, together with a peptide nanodisc and a membrane protein (d). The membrane protein (d) is reconstituted in the produced artificial biological membrane at a high probability, and retains lateral diffusivity.

### TECHNICAL FIELD

The present invention relates to an artificial biological membrane containing a membrane protein, and more specifically, to an artificial biological membrane containing a membrane protein retaining lateral diffusivity. The present invention also relates to a method of producing an artificial biological membrane containing a membrane protein.

The present application claims priority from Japanese Patent Application No. 2021-173293, which is incorporated herein by reference.

### BACKGROUND ART

To obtain a function and biocompatibility comparable to those of a biological membrane, many researches on the stabilization of a lipid membrane having the same structure as that of the biological membrane through polymerization with light or heat have been performed. A group of the inventors of the present invention has developed a patterned artificial biological membrane in which a photopolymerizable lipid membrane is polymerized by a photolithography technology to be combined with a natural lipid (Non Patent Literature 1, and Patent Literatures 1 and 2).

There is a disclosure of a nanofluid device including a nanogap structure-type substrate utilizing the technology of the patterned artificial biological membrane (Patent Literature 3). In the patent literature, there is a description of a substrate including, between a lipid membrane and a solid phase, a nanospace (nanogap structure) whose thickness is controlled to from 5 nm to 200 nm, and there is a description that the substrate may be utilized in, for example, a biosensor or biochip that can measure the physical properties of a biomolecule with high sensitivity. However, there is no description of a membrane containing a membrane protein.

The membrane protein plays an extremely important role in a living organism, and is also the main target molecule of a pharmaceutical or the like. However, the protein has a proper structure and a proper function only under the state of being incorporated into a lipid membrane. An attempt has heretofore been made to measure the physical properties and functions of the membrane protein through the production of an artificial biological membrane mimicking a biological membrane on the surface of a substrate serving as a support. However, it has been difficult to reconstitute the membrane protein in the artificial biological membrane while retaining intramembrane diffusivity, a function, and the like in the same manner as in the biological membrane.

There is a disclosure of a substrate in which a membrane fraction containing a membrane-bound cytochrome P450 (P450) is immobilized to a fluid lipid membrane region through utilization of the technology of a patterned artificial biological membrane (Patent Literature 4). The immobilization of the membrane-bound P450 to the membrane was performed by: diluting the protein-containing membrane fraction with a buffer containing 20% glycerol and 2-mercaptoethanol to (2-ME) an appropriate concentration; and adding the diluted fraction to a patterned simulated biological membrane. In the patent literature, there is a description of the immobilization of the P450 to the membrane, but there are no descriptions of fluidity and lateral diffusivity.

There is a disclosure of a technology including rapidly diluting a membrane protein (rhodopsin), which has been solubilized in a detergent, in an aqueous solution to introduce the protein into a patterned artificial biological membrane (Non Patent Literature 2). However, the technology has involved, for example, the following problems: (1) introduction efficiency is low (the density of the membrane protein is low); (2) the ratio of the membrane protein that is immobilized (free of lateral diffusivity) is high; (3) a defect or deformation may occur in the membrane owing to the action of the detergent; (4) the membrane protein nonspecifically adsorbs to the surface of a polymer lipid membrane; and (5) the membrane protein may aggregate to lose its activity at the time of the dilution of the detergent.

A nanodisc is a disc-shaped nanoparticle in which an amphipathic protein (membrane scaffold protein: MSP) surrounds a lipid bilayer in a belt fashion, and a disc-shaped nanoparticle artificially reconstituting a high-density lipoprotein (HDL) particle in blood has been generally known. There is a disclosure of a surface-active peptide with which a nanodisc can be prepared simply and in a short time period without use of any detergent (Patent Literature 5). In addition, there is a report of a lipid nanodisc surrounded by an amphipathic peptide (A18 peptide) instead of the MSP (Non Patent Literature 3). There is a report that the lipid nanodisc surrounded by the A18 peptide is reconstituted in a lipid membrane on the surface of a substrate (Non Patent Literatures 4 and 5). However, even when the lipid nanodisc surrounded by the amphipathic peptide is used, the ratio at which a membrane protein is reconstituted in the lipid membrane is low, and problems, such as the nonspecific adsorption and deactivation of the membrane protein, remain. In addition, the reconstituted membrane protein is immobilized and is hence free of lateral diffusivity. The membrane protein expresses a function, such as signal transduction or energy conversion, through the following: the protein two-dimensionally diffuses in a biological membrane to interact with a specific protein, to thereby form a composite, or two-dimensionally diffuses to accumulate in a specific lipid region. Accordingly, the development of an artificial biological membrane containing a membrane protein retaining lateral diffusivity has been desired.

### CITATION LIST

### Patent Literature

[PTL 1] JP 2004-309464 A (JP 4150793 B2)
[PTL 2] JP 2011-226920 A (JP 5532229 B2)
[PTL 3] JP 2016-125946 A
[PTL 4] JP 2008-187975 A
[PTL 5] JP 2017-039673 A

### Non Patent Literature

[NPL 1] Okazaki et al., Langmuir, 2009 Jan. 6; 25(1): 345-51
[NPL 2] Tanimoto et al., Biophys. J. 2015, 109, 2307-2316
[NPL 3] Midtgaard et al., Soft Matter, 2014, 10, 738-752
[NPL 4] Luchini et al., Anal. Chem. 2020, 92, 1081-1088
[NPL 5] Luchini et al., Journal of Colloid and Interface Science 585 (2021) 376-385

### SUMMARY OF INVENTION

### Technical Problem

The present invention relates to an artificial biological membrane containing a membrane protein. More specifically, an object of the present invention is to provide an artificial biological membrane containing a membrane protein retaining lateral diffusivity. Another object of the present invention is to provide a method of producing an artificial biological membrane containing a membrane protein.

### Solution to Problem

The inventors of the present invention have made extensive investigations with a view to achieving the above-mentioned objects, and as a result, have found an approach including: polymerizing a photopolymerizable lipid membrane by a photolithography technology to form a polymer lipid membrane; and combining the membrane with a fluid lipid membrane containing a peptide nanodisc containing a membrane protein to produce a patterned artificial biological membrane. The inventors have first recognized that according to the method of producing a patterned artificial biological membrane including using the peptide nanodisc containing the membrane protein in the patterned artificial biological membrane, the fluid lipid membrane containing the membrane protein extends from the peripheral region of the polymer lipid membrane, and the reconstituted membrane protein retains lateral diffusivity. Thus, the inventors have completed the present invention.

That is, the present invention includes the following.
1. A method of producing an artificial biological membrane, including a step of partially laminating a polymer lipid membrane (b) on a surface of a substrate (a), followed by lamination of a fluid lipid membrane (c) on the surface of the substrate (a), on which the polymer lipid membrane (b) is free from being laminated, together with a membrane protein (d) in a nanodisc containing an amphipathic peptide, wherein the membrane protein (d) is reconstituted under a state of retaining lateral diffusivity.
2. The method of producing an artificial biological membrane according to the above-mentioned item 1, wherein the step of partially laminating the polymer lipid membrane (b) on the surface of the substrate (a) is performed by: causing a lipid membrane of a polymerizable lipid (monomer) to adsorb to the surface of the substrate (a); and forming the polymer lipid membrane (b) through photopolymerization.
3. The method of producing an artificial biological membrane according to the above-mentioned item 1 or 2, wherein the method includes the following steps (1) to (4):
(1) a step of causing a lipid membrane of a polymerizable lipid (monomer) to adsorb to the surface of the substrate (a);
(2) a step of protecting part of the adsorbing lipid membrane from photoirradiation, followed by application of light to the polymerizable lipid (monomer) that is free from being protected from the photoirradiation to polymerize the monomer, to thereby subject the polymer lipid membrane (b) to pattern formation on the surface of the substrate (a);
(3) a step of removing the lipid membrane, which has been protected from a photopolymerization reaction in the step (2), from the surface of the substrate (a) with a detergent or an organic solvent; and
(4) a step of laminating the fluid lipid membrane (c) on a site of the surface of the substrate (a), from which the lipid membrane has been removed in the step (3), together with the membrane protein (d).
4. An artificial biological membrane, including: a substrate (a); and a lipid membrane laminated on a surface of the substrate (a), wherein the lipid membrane to be laminated includes a polymer lipid membrane (b) and a fluid lipid membrane (c) containing a membrane protein (d), and wherein the membrane protein (d) is in a state of retaining lateral diffusivity.
5. The artificial biological membrane according to the above-mentioned item 4, wherein the polymer lipid membrane (b) is partially laminated on the surface of the substrate (a), and wherein the fluid lipid membrane (c) containing the membrane protein (d) is laminated on the surface of the substrate (a) on which the polymer lipid membrane (b) is free from being laminated.
6. The artificial biological membrane according to the above-mentioned item 4 or 5, wherein the polymer lipid membrane (b) is a photopolymerizable lipid membrane.
7. The artificial biological membrane according to any one of the above-mentioned items 4 to 6, wherein the polymer lipid membrane (b) is subjected to pattern formation to be laminated on the surface of the patterned substrate (a), and wherein the fluid lipid membrane (c) containing the membrane protein (d) is laminated on the surface of the substrate (a) on which the polymer lipid membrane (b) is free from being laminated.
8. An artificial biological membrane, including: a substrate (a); and a lipid membrane laminated on a surface of the substrate (a), wherein the artificial biological membrane is produced by the production method of any one of the above-mentioned items 1 to 3, and wherein a membrane protein (d) is reconstituted under a state of retaining lateral diffusivity.
9. The artificial biological membrane according to any one of the above-mentioned items 4to 8, wherein the membrane protein (d) is one or two or more kinds of membrane proteins.
10. The artificial biological membrane according to the above-mentioned item 9, wherein at least one kind of the membrane protein (d) is a protein classified into a G protein-coupled receptor (GPCR).
11. A method of analyzing a function of a membrane protein (d), the method including using the artificial biological membrane of any one of the above-mentioned items 4 to 10.
12. A method of evaluating a substance targeting a membrane protein (d), the method including using the artificial biological membrane of any one of the above-mentioned items 4 to 10.
13. A device for an inspection concerning a membrane protein (d), the device including the artificial biological membrane of any one of the above-mentioned items 4 to 10.
14. A kit for an inspection concerning a membrane protein (d), the kit including the device for an inspection of the above-mentioned item 13 and a reagent required for the inspection of the membrane protein (d).

### Advantageous Effects of Invention

Heretofore, the lateral diffusivity and stability of a membrane protein have not been retained in an artificial biological membrane, and hence it has been unable to say that the membrane protein is sufficiently reconstituted in the artificial biological membrane. Meanwhile, it has been recognized that in the artificial biological membrane containing the membrane protein of the present invention, the reconstituted membrane protein retains lateral diffusivity, and hence has the same function as that of a biological membrane. Further, according to the method of producing an artificial biological membrane containing a membrane protein of the present invention, the polymer lipid membrane is subjected to pattern formation, and hence the fluid lipid membrane can be classified into corrals. Thus, the fluid lipid membrane changed in kind and concentration (density) of the membrane protein for each corral may be appropriately arranged in accordance with purposes.

When the artificial biological membrane containing the membrane protein of the present invention is used, the functional analysis of the membrane protein and the evaluation of a substance targeting the membrane protein can be performed by adjusting conditions, such as the kind and concentration (density) of the membrane protein, in an in vitro system in accordance with purposes.

### BRIEF DESCRIPTION OF DRAWINGS

FIG. 1 are views for illustrating the concepts of a method of producing an artificial biological membrane and pattern formation in the present invention. FIG. 1A is an illustration of a photopolymerizable lipid, and FIG. 1B is an illustration of a method of producing a patterned artificial biological membrane through photoirradiation. FIG. 1C shows the produced patterned artificial biological membrane. (Example 1)

FIG. 2 is a view for illustrating a method of producing an artificial biological membrane containing rhodopsin (Rh) as a membrane protein (d). The figure is an illustration of a formation process for a fluid lipid membrane (c) (bilayer) and the artificial biological membrane containing the membrane protein (d). (Example 1)

FIG. 3 are each a graph for showing a purification process for a peptide nanodisc. FIG. 3A is a graph for showing a purification process for a peptide nanodisc (Rh-ND) containing rhodopsin (Rh) to be used in the production of an artificial biological membrane, the process including using size exclusion chromatography (SEC). A peptide (A18 peptide) having amphipathicity and a lipid (POPC) were mixed in a solvent, and a dry film was formed in a glass vial, followed by the addition of an aqueous solution containing Rh that was solubilized. After that, a detergent was removed by the SEC, and thus the nanodisc was formed. Fractions 7 to 10 were Rh-containing nanodiscs (Rh-NDs), and fractions 15 and 16 were nanodiscs each formed only of the lipid (Empty-NDs). The Empty-NDs are secondarily obtained, and are not used at the time of the formation of a supported lipid bilayer (SLB) because the nanodiscs each contain, for example, a trace amount of Rh. FIG. 3B is a graph for showing a purification process for a peptide nanodisc containing only the lipid (Lipid-ND), the process including using the SEC. (Example 1)

FIG. 4 are photographs and graphs for showing the mode of the formation of the fluid lipid membrane (c) in the artificial biological membrane. A peptide nanodisc was introduced into a region (corral) surrounded by a grid-shaped polymer lipid membrane (b). FIG. 4A shows results obtained by observing the manner in which the fluid lipid membrane (c) is formed from a peripheral region (boundary region) with the polymer lipid membrane (b) with a fluorescence microscope, and FIG. 4B shows results obtained by converting the results of FIG. 4A into numerical values in terms of fluorescence intensity at up to 120 seconds. FIG. 4C shows results obtained by measuring the rates at which the fluid lipid membrane extends in the boundary region (peripheral region) with the polymer lipid membrane (b) and the central region of a corral free of the polymer lipid membrane (b). (Experimental Example 1)

FIG. 5 is a graph for showing results obtained by measuring the two-dimensional diffusion rates of respective Cy7-labeled rhodopsin (Cy7-NT-Rh) molecules reconstituted in corrals through single-molecule fluorescence tracking. A median for N=116 was 0.24 μm²s⁻¹. (Experimental Example 2)

FIG. 6 are photographs and a graph for showing the results of the recognition of the reconstitution of the Cy7-NT-Rh in the fluid lipid membrane (c) when a Cy7-NT-Rh-containing peptide nanodisc (Rh-ND) or detergent-treated Cy7-NT-Rh is used for the artificial biological membrane containing the membrane protein (d). FIG. 6A shows results obtained by observing the respective membrane proteins with a fluorescence microscope, and FIG. 6B shows results obtained by converting the reconstitution of the Cy7-NT-Rh into numerical values. (Comparative Example 1)

### DESCRIPTION OF EMBODIMENTS

The present invention relates to an artificial biological membrane containing a membrane protein, and more specifically, to an artificial biological membrane obtained by laminating a lipid membrane on the surface of a substrate, the artificial biological membrane being characterized in that the lipid membrane to be laminated is a lipid membrane containing a polymer lipid membrane and a fluid lipid membrane containing a membrane protein. The polymer lipid membrane is partially laminated on the surface of the substrate, and the fluid lipid membrane containing the membrane protein is laminated on the surface of the substrate on which the polymer lipid membrane is free from being laminated. The polymer lipid membrane is preferably a photopolymerizable lipid membrane.

The term “artificial biological membrane” as used herein refers to a biological membrane that is artificially produced while retaining the basic physical properties of the biological membrane. The biological membrane has such a bilayer structure that lipid molecules are arranged to form two layers on its front and rear surfaces, and the membrane retains the basic physical property (fluidity) by which the molecules can laterally diffuse. The artificial biological membrane of the present invention is a lipid bilayer produced on the surface of the substrate serving as a support. The artificial biological membrane on the surface of the substrate is also referred to as “supported lipid bilayer (SLB).”

The artificial biological membrane of the present invention is suitably a patterned artificial biological membrane. The term “patterned artificial biological membrane” as used herein refers to an artificial biological membrane in which the polymer lipid membrane free of fluidity is arranged in a patterned shape on the surface of the substrate, and the fluid lipid membrane is arranged on the surface of the substrate on which the polymer lipid membrane is not arranged. A method for pattern formation is described later.

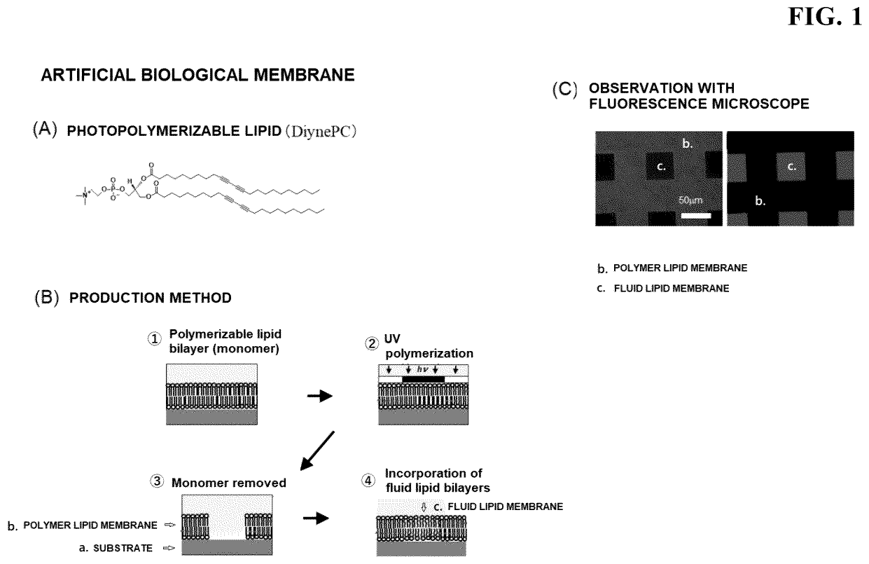

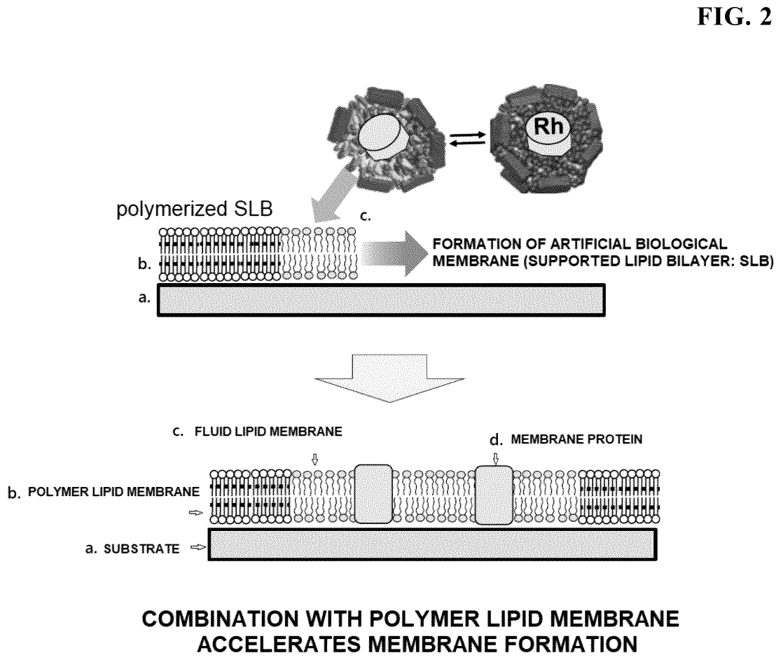

The “substrate” as used herein only needs to be a solid on which the biological membrane is supported and to which the biological membrane can be bonded through a physical interaction or a chemical bond, and hence the substrate is not particularly limited (see FIG. 1 and FIG. 2). Examples of a material for the substrate include glass, quartz, plastic, a ceramic, and a metal, and the material may be appropriately selected therefrom. Preferred examples thereof include glass, quartz, a polymeric elastomer, and silicon oxide. The polymeric elastomer is a collective term for industrial materials each having rubber-like elasticity, and specific examples thereof include styrene-based, olefin-based, vinyl chloride-based, urethane-based, and amide-based elastomers. More specific examples thereof include silicon elastomers such as polydimethylsiloxane (PDMS). In addition, the shape may be a flat plate or a curved shape.

The “lipid membrane” as used herein may be derived from a natural or synthetic origin, and is, for example, a lipid membrane having a phospholipid such as a phosphatidylcholine. Although the lipid membrane may be a lipid bilayer or a lipid monolayer, the membrane is particularly suitably a lipid bilayer (see FIG. 1 and FIG. 2).

The term “polymer lipid membrane” as used herein refers to a polymer lipid membrane having the same membrane structure as that of the biological membrane, and the lipid membrane is preferably a photopolymerizable lipid membrane, but may be a simulated biological membrane. The photopolymerizable lipid membrane only needs to be a lipid having at least one photopolymerizable group, and is hence not particularly limited. Examples of the photopolymerizable group include a double bond, a triple bond, an epoxy salt, and an a, B-unsaturated carbonyl group. Of those, a double bond or a triple bond is preferred. One lipid molecule contains preferably 1 to 10, more preferably 2 to 6 photopolymerizable groups. Specifically, a lipid membrane described in Patent Literature 1, which is formed of a polymer bilayer structure, is suitable. A polymerizable lipid is, for example, a phospholipid, and specifically, one or two acyl groups of a phosphatidylcholine, a phosphatidylethanolamine, phosphatidylinositol, or phosphatidylserine are each preferably a group derived from a linear or branched aliphatic carboxylic acid having typically 6 to 30, preferably 6 to 24, more preferably 12 to 22 carbon atoms, the acid containing at least one photopolymerizable group. Although the lipid is preferably a glycerophospholipid having two acyl groups, the following phospholipid may be used in combination with the glycerophospholipid: one acyl group of the phospholipid is hydrolyzed, and hence one acyl group (having a photopolymerizable group) remains.

Reference may be made to Patent Literature 1 or 2 for the structures of the photopolymerizable group and a lipid monomer. With regard to the lipid membrane, when a reactive group such as a primary amine having chemical reactivity with the hydrophilic moiety of a photopolymerizable lipid molecule is introduced, a bio-related molecule can be chemically bonded to the surface of the polymer lipid membrane to produce an artificial biological membrane having a biological function. The hydrophilic moiety of a photopolymerizable phospholipid 1,2-bis(10,12-tricosadiynoyl)-sn-glycero-3-phosphochorine (hereinafter referred to as “DiynePC”) is a chemically inert choline group.

In DiynePC, a diacetylene group may be present at any position of its hydrocarbon chain. For example, the group may be present at any one of the terminal sides thereof. The diacetylene group is incorporated into a fatty acid having 10 to 30, preferably 14 to 28, more preferably 18 to 26 carbon atoms, and the fatty acid forms an ester with two glycerol groups to provide a photopolymerizable lipid having a diacetylene group. When a lipid membrane formed of a monomer of the photopolymerizable lipid having a diacetylene group is formed on the surface of the substrate, to achieve a crystal state in which lipid molecules are regularly oriented, the temperature of the substrate is suitably set to be lower than the phase transition temperature of the monomer of the photopolymerizable lipid having a diacetylene group by 5° C. or more. For example, the phase transition temperature of the lipid membrane of the photopolymerizable phospholipid DiynePC is 38° C., and hence the temperature of the substrate is suitably 33° C. or less, more suitably 10° C. or less. The phase transition temperature varies depending on the photopolymerizable lipid having a diacetylene group, and hence the substrate is used at a preferred temperature in accordance with the lipid.

The term “fluid lipid membrane” as used herein refers to a lipid membrane having the same membrane structure as that of the biological membrane and having fluidity (molecular lateral diffusivity) as in a cell membrane. The term “lateral diffusivity” as used herein is used in a meaning generally described as a membrane property, and specifically refers to the property by which a lipid molecule for forming a membrane performs a diffusive motion in the membrane. In a lipid bilayer, a phospholipid molecule is free of any covalent bond, and hence can perform various molecular motions. Out of motions in which the positions of the lipid molecules are exchanged with each other (diffusive motions), a motion in which the molecules move in one and the same monolayer in a lateral direction is referred to as “lateral diffusive motion.” The cell membrane is not a static structural body that merely separates the inside and outside of a cell from each other, and the membrane plays functions important for the cell, such as: a function of passing a low-molecular weight molecule such as an ion through a specific channel; a function of receiving a signal from the outside of the cell through a receptor; and a function of taking in part of the cell membrane, followed by its transportation into the cell. In addition, in a hydrophilic substrate, a hydration layer is present between the lipid membrane adsorbing to the substrate through a physical interaction and the substrate, and hence the lipid molecules to be formed on the hydrophilic substrate can also laterally diffuse as in the biological membrane.

The term “membrane protein” in the present invention refers to a protein adhering to each of the biological membranes of cells and intracellular organelles in all living organisms ranging from microorganisms to animals and plants. The membrane proteins are classified into an intrinsic membrane protein and a superficial membrane protein. Further, the membrane proteins are classified into a plurality of types depending on the direction in which the proteins are each inserted into a membrane, the presence or absence of a signal peptide, and the positions of transmembrane domains in the proteins. Examples of the function of the membrane protein include: the conveyance of a substance required for the maintenance of a life, such as an ion or a nutrient; and a high-sensitivity sensor for extracellular information called a sensory receptor for a hormone, light, heat, sound, or the like. The membrane proteins are specifically classified into, for example, a G protein-coupled receptor (GPCR), an ion channel, a membrane-bound enzyme, and a transporter. Examples of the GPCR include receptor families, such as a rhodopsin-like receptor (class A), a secretin-like receptor (class B), a metabotropic glutamate receptor (class C), and Frizzled/Smoothened (class F). Specific examples thereof include rhodopsin, an adrenaline receptor, a histamine H1 receptor, a histamine H2 receptor, a serotonin receptor, and a dopamine receptor. Other examples of the membrane protein include cytochrome P450 and an ATP synthase.

The artificial biological membrane containing the membrane protein of the present invention may be produced by: partially laminating the polymer lipid membrane on the surface of the substrate; and laminating, on the surface of the substrate on which the polymer lipid membrane is not laminated, the fluid lipid membrane together with the membrane protein.

In the production of the artificial biological membrane of the present invention, the arrangement of the polymer lipid membrane and the fluid lipid membrane may be arbitrarily determined by using a photolithography technology. A lipid membrane formed of a bilayer structure may be formed by causing a photopolymerizable polymer lipid membrane to adhere onto the substrate in accordance with a photopolymerization method described in, for example, Patent Literature 2 (JP 5532229 B2). In the case where the polymer lipid membrane is caused to adhere onto the substrate in accordance with the photopolymerization method to form the lipid membrane formed of the bilayer structure, the polymer lipid membrane may be formed by: protecting part of the adsorbing polymerizable lipid (monomer) from photoirradiation; and applying light to the polymerizable lipid (monomer) that is not protected from the photoirradiation to polymerize the monomer. In this case, a site to be protected from the photoirradiation may be arbitrarily determined, and hence the shape of the site to be subjected to the photoirradiation may be patterned. The term “pattern formation” as used herein means that the shape of the site to be subjected to the photoirradiation is patterned. Although the patterned shape is specifically, for example, a grid-shaped pattern illustrated in FIG. 1B, the shape is not limited to such shape, and may be arbitrarily determined in accordance with purposes. The artificial biological membrane containing the membrane protein in which the polymerized polymer lipid membrane is subjected to pattern formation may be produced by, for example, the following method. Thus, the artificial biological membrane containing the membrane protein in a hybrid membrane, which is obtained by combining the polymer lipid membrane and the fluid lipid membrane with each other, can be produced on the substrate (see FIG. 1B).

The artificial biological membrane containing the membrane protein of the present invention may be specifically produced by a method including the following steps (1) to (4). The method is more specifically described below on the basis of symbols illustrated in FIG. 1B or FIG. 2.
(1) A step of causing a lipid membrane of a polymerizable lipid (monomer) to adsorb to the surface of the substrate (a).
(2) A step of protecting part of the adsorbing lipid membrane from photoirradiation, followed by application of light to the polymerizable lipid (monomer) that is free from being protected from the photoirradiation to polymerize the monomer, to thereby subject the polymer lipid membrane (b) to pattern formation on the surface of the substrate (a).
(3) A step of removing the lipid membrane, which has been protected from a photopolymerization reaction in the step (2), from the surface of the substrate (a) with a detergent or an organic solvent.
(4) A step of laminating the fluid lipid membrane (c) on a site of the surface of the substrate (a), from which the lipid membrane has been removed in the step (3), together with the membrane protein (d).

In the above-mentioned steps (2) and (3), a lipid membrane formed of a bilayer structure may be arbitrarily subjected to pattern formation by causing the photopolymerizable polymer lipid membrane (b) to adhere onto the substrate in accordance with a photopolymerization method described in, for example, Patent Literature 3 (JP 5532229 B2) or Non Patent Literature 4 (Langmuir 2013, 29(8), 2722-2730).

In the above-mentioned step (4), the lamination of the fluid lipid membrane (c) on the surface of the substrate (a) together with the membrane protein (d) is achieved by dropping a fluid lipid suspension containing the membrane protein (d) onto the surface of the substrate (a). In this case, the membrane protein (d) is suitably dropped together with the fluid lipid suspension under a state in which the protein is incorporated into a nanodisc containing an amphipathic peptide.

The term “nanodisc containing an amphipathic peptide” as used herein refers to a disc-shaped nanoparticle containing the amphipathic peptide instead of an amphipathic protein (membrane scaffold protein: MSP). When the membrane protein (d) is handled together with the nanodisc containing the amphipathic peptide, the membrane protein (d) separated from the biological membrane can be stably used. However, as described in the section “Background Art,” even when the lipid nanodisc surrounded by the amphipathic peptide is reconstituted in the lipid membrane on the surface of the substrate (a), heretofore, the membrane protein (d) has been immobilized and hence has been free of lateral diffusivity (Non Patent Literatures 3 to 5).

A disc-shaped aggregate (nanodisc) may be formed by solubilizing a phospholipid liposome with the amphipathic peptide that may be utilized in the nanodisc. When a molar ratio between a phospholipid and the amphipathic peptide is from 33:1 to 23:1, the edge of a disc-shaped lipid bilayer is covered with the hydrophobic side surface of the peptide, and hence a nanodisc in a quasi-stable state can be formed. When the membrane protein (d) such as rhodopsin (Rh) is incorporated into such nanodisc, the object can be achieved by: mixing the membrane protein solubilized with the detergent, the lipid, and the amphipathic peptide; and removing the detergent from the solution. The amino acid sequence of the amphipathic peptide in the nanodisc of the present invention is, for example, an amino acid sequence set forth in SEQ ID: 1 below. However, the peptide only needs to be an amphipathic peptide that may be utilized in a nanodisc, and hence its amino acid sequence is not particularly limited.

### A18 Peptide: DWLKAFYDKVAEKLKEAF (SEQ ID: 1)

Reference may be made to FIG. 1 for the mode of the pattern formation of the artificial biological membrane in the present invention. The artificial biological membrane of the present invention is produced by laminating the fluid lipid membrane (c) in corrals surrounded by the polymer lipid membrane (b) together with the membrane protein (d). The kind and concentration of the membrane protein (d) may be appropriately changed.

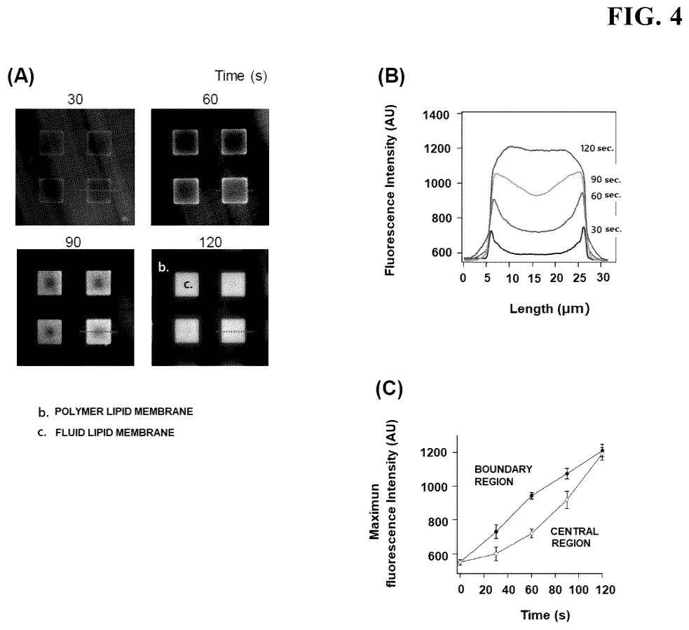

Specifically, the nanodiscs described in Non Patent Literatures 3 to 5 described in the section “Background Art” may each be used as the “nanodisc containing an amphipathic peptide” in this description. In this description, the membrane protein (d) in the nanodisc containing the amphipathic peptide (hereinafter simply referred to as “peptide nanodisc”) is laminated on the surface of the substrate (a) together with the fluid lipid membrane (c). The lamination on the surface of the substrate (a) may be performed by: mixing a peptide nanodisc suspension containing the membrane protein (d) and a peptide nanodisc suspension free of the membrane protein (d) at an arbitrary ratio; and dropping the mixed liquid onto the surface of the substrate (a) to form the protein-incorporated fluid lipid membrane (c). The fluid lipid membrane (c) is formed so as to extend from the peripheral region of the polymer lipid membrane (b) (see FIG. 2 and FIG. 4). The foregoing suggests that the polymer lipid membrane (b) formed in advance on the surface of the substrate (a) plays an important role in the reconstitution of the fluid lipid membrane (c).

That is, when the polymer lipid membrane (b) is not present on the surface of the substrate (a), the formation of the fluid lipid membrane (c) requires a time period longer than that in a state in which the polymer lipid membrane (b) is present. As a result, the nanodisc containing the membrane protein (d) is immobilized to the substrate (a), and hence the fluidity of the protein is lost. When the fluid lipid membrane (c) is formed so as to extend from the peripheral region of the polymer lipid membrane (b), the fluidity of the membrane protein (d) is retained, and the lateral diffusivity thereof is also retained.

In the artificial biological membrane containing the membrane protein (d) produced in the present invention, the fluid lipid membrane (c) can be classified into corrals by the pattern formation of the polymer lipid membrane (b). The membrane protein (d) may be arbitrarily arranged in the fluid lipid membrane (c) classified for each corral while its kind and concentration (density) are changed. Further, the plurality of kinds of membrane proteins (d) may be introduced in combination. The interaction of a substance targeting the introduced membrane protein (d) may be measured. The use of such artificial biological membrane enables the analysis of the function of the membrane protein (d) and the evaluation of the substance targeting the membrane protein (d). The substance targeting the membrane protein (d) only needs to be a substance capable of interacting with the membrane protein (d), and is hence not particularly limited. However, examples thereof include a drug such as a pharmaceutical, a component in food, and a biomarker in a living organism. Heretofore, the lateral diffusivity of a membrane protein in an artificial biological membrane has not been retained, and hence it has been unable to say that the function of the membrane protein is sufficiently reflected on the artificial biological membrane. However, when the artificial biological membrane containing the membrane protein of the present invention is used, the functional analysis of the membrane protein and the evaluation of the substance targeting the membrane protein can be performed in an in vitro system. The present invention covers a method of analyzing the function of a membrane protein and a method of evaluating a substance targeting the membrane protein each including using the artificial biological membrane of the present invention.

The present invention also covers a device for an inspection concerning the membrane protein (d), the device including the above-mentioned artificial biological membrane. The device for an inspection may be an artificial biological membrane itself obtained by laminating a lipid membrane on the surface of the substrate (a), the artificial biological membrane being characterized in that the lipid membrane to be laminated includes the polymer lipid membrane (b) and the fluid lipid membrane (c) containing the membrane protein (d), and the membrane protein (d) is in the state of retaining its lateral diffusivity. Alternatively, the device may be a device in which the artificial biological membrane is placed on a tray or the like so as to be portable and applicable to a measuring instrument. The term “inspection concerning the membrane protein (d)” as used herein means an inspection concerning the functional analysis of the membrane protein (d) and the evaluation of a substance targeting the membrane protein (d). In the device for an inspection concerning the membrane protein (d), the fluid lipid membrane (c) can be classified into corrals by the pattern formation of the polymer lipid membrane (b) as described above. The membrane protein (d) may be arbitrarily arranged in the fluid lipid membrane (c) classified for each corral while its kind and concentration (density) are changed. Further, the plurality of kinds of membrane proteins (d) may be introduced in combination. Thus, there can be provided a device for an inspection in which the shape of the pattern, the kind of the membrane protein, the concentration of the membrane protein, and the like are appropriately customized in accordance with the purpose of the inspection.

The present invention also covers a kit for an inspection concerning the membrane protein (d), the kit including at least the device for an inspection concerning the membrane protein (d), and further including a reagent and/or an instrument required for the inspection of the membrane protein (d). Examples of the “reagent required for the inspection of the membrane protein (d)” as used herein include: a water-soluble reagent required for the function of the membrane protein such as a coenzyme; a buffer reagent for sample dilution; and a marker reagent for an inspection. However, the reagent is not limited thereto, and various reagents that may be used as kits are given as examples thereof. In addition, examples of the “instrument required for the inspection of the membrane protein (d)” include: an instrument for introducing a sample, a reagent, or the like into the device for an inspection to perform solution exchange; an instrument: for performing measurement; and an instrument for protecting the device for an inspection. However, the instrument is not limited thereto, and various instruments that may be used as kits are given as examples thereof.

### EXAMPLES

To deepen the understanding of the present invention, the contents of the present invention are described more specifically by way of Example, Experimental Examples, and Comparative Example while reference is made to the contents illustrated in the drawings. However, it is obvious that the present invention is not limited to these examples.

### (Example 1) Production of Patterned Artificial Biological Membrane Containing Membrane Protein (d)

In this example, a method of producing a patterned artificial biological membrane containing the membrane protein (d) is described. A lipid bilayer was prepared through the immobilization of the polymer lipid membrane (b) to the surface of the substrate (a) by a vesicle fusion method. In this example, a glass substrate was used as the substrate (a), and rhodopsin (Rh) was used as the membrane protein (d).

### 1-1. Preparation of Polymer Lipid Membrane (b)

Ultrapure water was added to 10 mg of a photopolymerizable lipid (DiynePC) so that its final concentration became 3 mM. Further, a solution (0.15 mM) of a detergent (DHPC-C6) was added and mixed into the mixture for accelerating vesicle fusion. Thus, a DiynePC suspension was prepared. The DiynePC suspension was frozen with liquid nitrogen, and was thawed at 60° C. The operation was repeated five times. After the freezing and the thawing, the resultant was homogenized with an ultrasonic homogenizer (Branson Sonifier™ 150, Emerson Electric Co.) at 60° C. and an output setting of 30 W for 30 seconds twice. The produced DiynePC suspension was deposited on the substrate (a) washed on ice, and a monomer bilayer was immediately cooled.

The DiynePC lipid bilayer adhering to the surface of the substrate (a) was held in pure water. Before the performance of the photopolymerization of the lipid bilayer, an aqueous solution purged with an inert gas (argon or nitrogen) for removing oxygen in the solution was circulated with a pump. After sufficient deaeration, the substrate (a) having laminated thereon the DiynePC lipid bilayer was subjected to UV irradiation with a deep UV exposure lamp (USHIO SP-9, Ushio Inc.) having a strong bright line at a wavelength of 300 nm or less. At that time, light having a wavelength of about 250 nm, which was most effective for a photochemical reaction, was selectively applied by using an interference filter or an interference mirror for a laser. In addition, to transfer a specific pattern, the substrate (a) was horizontally placed so that the surface of the lipid bilayer laminated thereon faced upward, and a mask for shielding light was mounted thereon (see FIG. 1B). After the photoirradiation, the resultant was bathed in a detergent solution (sodium dodecyl sulfate: SDS, 100 mM) at 30° C. for 30 minutes, and was then washed with pure water 10 times. Thus, the lipid bilayer in the site irradiated with the light was laminated as the polymer lipid membrane (b) on the surface of the substrate (a), and the bilayer in the site that had not been irradiated with the light was washed and removed. Thus, the polymer lipid membrane (b) was subjected to pattern formation (see FIG. 1B and FIG. 1C).

### Examples of Conditions Suitable for Photopolymerization

Temperature: from 0° C. to 4° C. (ice bath)
Irradiation wavelength: a deep UV exposure lamp (USHIO SP-9, Ushio Inc.), 300 nm or less
Intensity of light to be applied: 160 mW/cm² (254 nm)
Irradiation time: 100 seconds
Quantity of light to be applied: 16.0 J/cm²
   1-2. Preparation of Peptide Nanodisc containing Rhodopsin (Rh) (Cy7-Rh-ND)

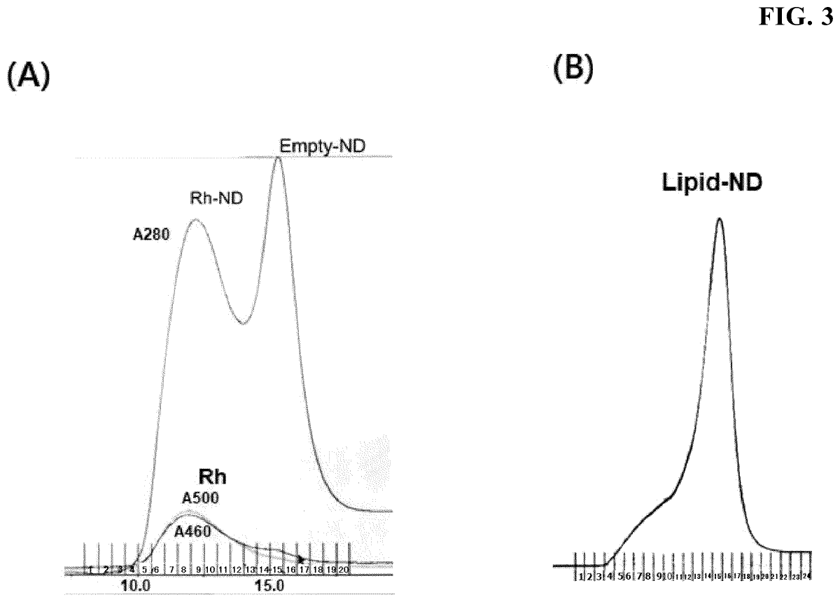

A lipid nanodisc (Cy7-Rh-ND) required for the formation of the fluid lipid membrane (c) containing rhodopsin (hereinafter also simply referred to as “Rh”) was prepared by the following method. Rh was obtained from a bullfrog photoreceptor. A photoreceptor outer segment membrane (90% or more of its proteins were accounted for by Rh) was subjected to hypotonic treatment so that the proteins except Rh were removed, followed by the oxidation of the N-terminal sugar chain of Rh through treatment with 10 mM periodic acid. Cy7-hydrazide was caused to react with the oxidized sugar chain to fluorescently label Rh. The outer segment membrane containing the fluorescently labeled Rh (Cy7-NT-Rh) was solubilized with 50 mM octyl glucoside in the coexistence of 0.1% azolectin. The Cy7-NT-Rh was purified by two-stage size exclusion chromatography (SEC) (Superose™ 12 3.2×300, Pharmacia; equilibrated with a buffer containing 0.1% azolectin and 50 mM octyl glucoside). 70 Micrograms of palmitoyl oleoyl phosphatidylcholine (POPC: 1 mM), 0.3 μg of a fluorescent lipid (NBD-PE), and 0.1 mg of an amphipathic peptide (A18 peptide: SEQ ID: 1) were mixed in methanol, and the mixture was dried in a small brown bottle in argon and a vacuum. Thus, a peptide lipid film was produced. 100 Microliters of a buffer A (100 mM NaCl, 10 mM CaCl₂, and 10 mM Tris-HCl, pH: 7.5) containing 50 mM octyl glucoside and 0.1% azolectin was added to the peptide lipid film, and vortex treatment and ultrasonic treatment were performed under an argon environment to dissolve the film therein. 50 Micrograms of the solubilized Cy7-NT-Rh (50 μl) was added to the solution, and the mixture was incubated at room temperature for 30 minutes. After that, an aggregate was removed with a 0.22-micrometer filter, and a Cy7-NT-Rh nanodisc (Cy7-Rh-ND) and a peptide nanodisc free of Rh (Empty-ND) were separated and purified by SEC (Superdex™ 200 2/300, Pharmacia) (FIG. 3A).

### 1-3. Preparation of Lipid Nanodisc (Lipid-ND) Required for Formation of Fluid Lipid Membrane (c)

A lipid nanodisc (Lipid-ND) suspension for laminating the fluid lipid membrane (c) was prepared by the following method. A lipid suspension (POPC (1 mM) and NBD-PE (1 mol %)) formed of POPC (1 mg) and a fluorescent lipid (NBD-PE) (1 mol %) was dissolved in chloroform, and the solution was loaded into a small brown bottle. A N₂ gas was blown onto the solution to dry chloroform for 15 minutes. 0.5 Milliliter of the buffer A was added to Rh, and the mixture was subjected to simple ultrasonic treatment. After that, the mixture was left at rest at 37° C. overnight. The operation results in the formation of a lipid nanodisc including only the lipid. The lipid nanodisc was purified by SEC (Superdex™ 200 10×300, Pharmacia) for the purpose of removing an excess peptide (FIG. 3B).

### 1-4. Preparation of Patterned Artificial Biological Membrane Containing Rh

The mixture of the Cy7-Rh-ND prepared in the section 1-2 and the Lipid-ND prepared in the section 1-3 was dropped onto the surface of the substrate (a) in each of corrals surrounded by the patterned polymer lipid membrane (b) produced in the section 1-1 to cause the spontaneous formation of a SLB (the fluid lipid membrane (c)) (see FIG. 1B and FIG. 2). The fluid lipid membrane (c) was formed in 120 seconds after the dropping (FIG. 4A).

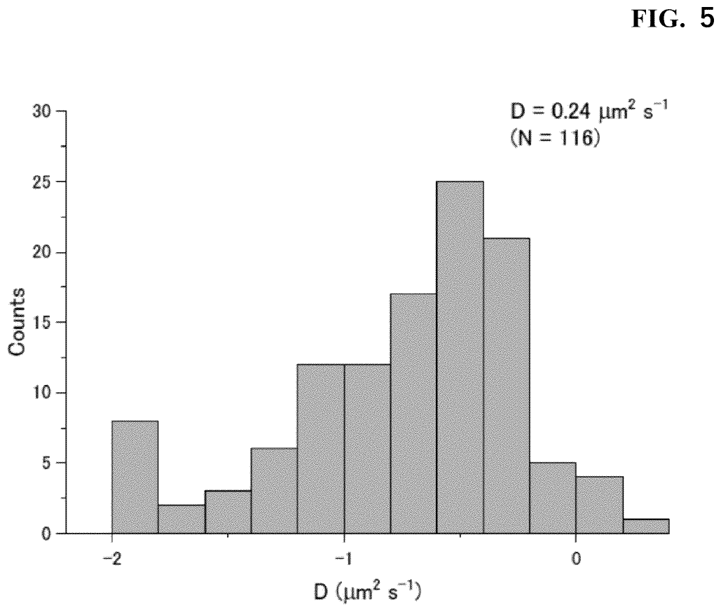

The Cy7-Rh-ND and the Lipid-ND were mixed at a ratio of 60:1, and the mixture was added to the substrate (a) mounted with the patterned polymer lipid membrane (b). The resultant was incubated for 15 minutes at 25° C., and then a redundant peptide nanodisc that had not adsorbed to the surface of the substrate (a) was removed by exchanging the solution with the buffer A. Further, the solution was exchanged with simulated physiological saline (potassium gluconate (98.7 mM), KCl (2.5 mM), magnesium chloride (1 mM), and HEPES (10 mM)) containing an oxygen-removed buffer for microscopic observation (glucose (4.5 mg/ml), glucose oxidase (216 μg/ml), and catalase (36 μg/ml)) suitable for preventing the bleaching of a fluorescent lipid, and the Cy7-NT-Rh in the patterned artificial biological membrane was subjected to fluorescence single-molecule observation with an inverted fluorescence microscope (Olympus IX73, Olympus Corporation). Fluorescence was observed by exciting the polymer lipid membrane (b), the fluid lipid membrane (c), and the Cy7-NT-Rh at wavelengths different from each other (see FIG. 4 and FIG. 5). The fluorescence of the fluid lipid membrane (c) was observed by exciting NBD-PE at an excitation wavelength of from 470 nm to 495 nm and a fluorescence wavelength of from 510 nm to 550 nm. The fluorescence of the Cy7-NT-Rh was observed at an excitation wavelength of 750 nm and a fluorescence wavelength of from 780 nm to 840 nm.

### (Experimental Example 1) Observation of Mode of Formation of Fluid Lipid Membrane

The mode of the formation of the fluid lipid membrane (c) produced in Example 1 was observed. The fluid lipid membrane (c) was formed and extended from the peripheral region of the polymer lipid membrane (b). Fluorescence intensities in the boundary region of the fluid lipid membrane with the polymer lipid membrane (b) and a central region in a corral thereof were measured. A change in fluorescence intensity along a section of the lower right corral of each pattern shown in FIG. 4A with time was observed. As a result, it was recognized that membrane formation was accelerated with the lapse of a time period as short as up to 120 seconds (FIG. 4B). Membrane formation rates in the respective boundary region and central region were compared to each other. As a result, it was recognized that the membrane formation rate in the boundary region was faster (FIG. 4C). Those results suggest that the polymer lipid membrane (b) formed on the surface of the substrate (a) plays an important role in the reconstitution of the membrane protein (d) in the fluid lipid membrane (c).

### (Experimental Example 2) Lateral Diffusivity of Reconstituted Cy7-NT-Rh Molecule

It was observed that substantially all the Cy7-NT-Rh molecules reconstituted in the corrals laterally diffused. The determination of the two-dimensional diffusion rates of the respective molecules by single-molecule fluorescence tracking provided a distribution shown in FIG. 5 whose median was 0.24 μm²s⁻¹ (N=116). The median diffusion rate was substantially the same as that of a detergent-treated Rh-containing patterned artificial biological membrane produced by a rapid dilution method described in Non Patent Literature 2.

### (Comparative Example 1) Comparison Between Incorporation of Membrane Protein with Peptide Nanodisc and Incorporation of Membrane Protein by Rapid Dilution of Detergent

The reconstitution of Rh in the fluid lipid membrane (c) containing a Cy7-NT-Rh-containing peptide nanodisc (Rh-ND) produced by the same approach as that of Example 1 or the Cy7-NT-Rh treated with a detergent (octyl glucoside: OG) was recognized. In addition, the lateral diffusivity of the reconstituted Rh was recognized.

A lipid suspension for laminating the fluid lipid membrane (c) to be used in this comparative example was prepared by the following method. A lipid suspension (POPC (1 mM) and NBD-PE (1 mol %)) formed of palmitoyl oleoyl phosphatidylcholine (POPC: 1 mM) and a fluorescent lipid (NBD-PE: N-(7-nitrobenz-2-oxa-1,3-diazol-4-yl)-1,2-dihexadecanoyl-sn-glycero-3-phosphoethanolamine, triethylammonium salt) (1 mol %) was dissolved in chloroform, and the solution was loaded into a round-bottom flask. A N₂ gas was blown onto the solution to dry chloroform for 15 minutes. Further, vacuum drying was performed for 4 hours to prepare a lipid film (in which many lipid bilayers were laminated) at the bottom of the round-bottom flask. Phosphate-buffered saline (PBS) was loaded into the round-bottom flask containing the lipid film, and the mixture was left at rest overnight so that the lipid film was swollen. Thus, a lipid membrane suspension was produced.

### Preparation of Detergent (OG)-Treated Rh-Containing Patterned Artificial Biological Membrane

A Cy7-NT-Rh-containing patterned artificial biological membrane was prepared by a rapid dilution method. The lipid suspension (POPC (1 mM) and NBD-PE (1 mol %)) prepared in the foregoing was homogenized with an ultrasonic homogenizer (Branson Sonifier™ 150, Emerson Electric Co.) at 60° C. and an output setting of 3 W for 30 seconds three times. The lipid suspension produced in the foregoing was dropped onto the site from which the polymer lipid had been removed because of the absence of photoirradiation out of the patterned polymer lipid membrane (b) produced in the section 1-1 of Example 1 described above to cause the spontaneous formation of the SLB (see FIG. 1B and FIG. 2). The Cy7-NT-Rh produced by the same approach as that of the section 1-2 of Example 1 described above was solubilized with OG (50 mM), and was added to an aqueous phase in the upper region of the SLB so that its final concentration became 3 mM or less. The aqueous phase was incubated at 25° C. for 10 minutes while being stirred, followed by rapid dilution. The Cy7-NT-Rh that had not been reconstituted in the SLB was removed by rinsing with the buffer A.

### (Results of Comparative Experiment)

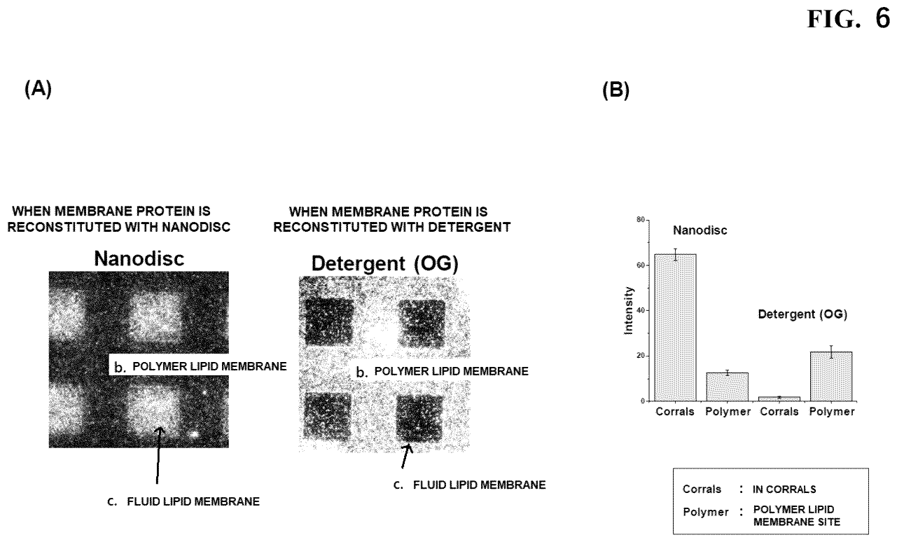

The nanodisc-treated Rh-containing patterned artificial biological membrane (Nanodisc) prepared in Example 1 and the detergent (OG)-treated Rh-containing patterned artificial biological membrane (OG) were compared to each other by using a fluorescence microscope image. It was recognized that in the case of the Nanodisc, a larger number of Cy7-NT-Rh molecules were reconstituted in the corrals as compared to that in the case where the detergent (OG) was used (FIG. 6A). Fluorescence intensities in the corrals and the site (Polymer) of the polymer lipid membrane (b) were compared to each other. As a result, it was recognized that in the case of the Nanodisc, the amount of the Cy7-NT-Rh molecules to be introduced into the corrals increased to be about 35 times as large as that in the case where the detergent (OG) was used, and the amount of the molecules nonspecifically adsorbing to the polymer lipid membrane reduced to be about 0.6 times as large as that in the case where OG was used (FIG. 6B).

When a membrane protein solubilized with a detergent is introduced into an artificial biological membrane, for example, the following problems occur: (1) introduction efficiency is low (the density of the membrane protein is low); (2) the ratio of the membrane protein that is immobilized (free of lateral diffusivity) is high; (3) a defect or deformation may occur in the membrane owing to the action of the detergent; (4) the membrane protein nonspecifically adsorbs to the surface of a polymer lipid membrane (FIG. 6B); and (5) the membrane protein may aggregate to lose its activity at the time of the dilution of the detergent.

Meanwhile, the patterned artificial biological membrane containing the membrane protein produced by the method of the present invention is excellent because of the following reasons: the efficiency with which the membrane protein is introduced is high (the density of the membrane protein is high); the membrane protein stably has lateral diffusivity; and the extent to which the membrane protein nonspecifically adsorbs to the surface of the polymer lipid membrane is low. Further, the detergent and the like are removed in a production process for the patterned artificial biological membrane containing the membrane protein of the present invention, and hence adverse effects, such as a defect and deformation in the membrane, and the aggregation of the membrane protein, are alleviated. It was recognized from the foregoing that the nanodisc-treated membrane protein-containing patterned artificial biological membrane of the present invention was superior to the detergent-treated membrane protein-containing patterned artificial biological membrane.

### INDUSTRIAL APPLICABILITY

As described in detail above, the membrane protein reconstituted in the artificial biological membrane containing the membrane protein of the present invention retains lateral diffusivity, and hence the artificial biological membrane of the present invention has the same function as that of a biological membrane. Further, according to the method of producing an artificial biological membrane containing a membrane protein of the present invention, the patterning of the polymer lipid membrane enables the fluid lipid membrane to be classified into corrals. Thus, the fluid lipid membrane changed in kind and concentration (density) of the membrane protein for each corral may be appropriately arranged in accordance with purposes.

The membrane protein plays an extremely important role in a living organism, and is also the main target molecule of a pharmaceutical or the like. However, the protein has a proper structure and a proper function only under the state of being incorporated into a lipid membrane. Accordingly, it has been difficult to perform sufficient functional analysis in an in vitro system. When the artificial biological membrane containing the membrane protein of the present invention is used by being utilized in, for example, a device or kit for an inspection, the functional analysis of the membrane protein and the evaluations of substances each targeting the membrane protein, the substances including a drug such as a pharmaceutical, food, and various substances that may each interact with the membrane protein, can be performed by adjusting conditions in the in vitro system in accordance with purposes. Thus, although heretofore, it has been possible to perform the evaluations only in an in vivo system, and it has been difficult to perform the evaluations unless an animal except a human is used, the evaluations can be simply performed by changing the conditions in a human system, and hence industrial applicability can be sufficiently expected in an economic sense.

1. A method of producing an artificial biological membrane, comprising a step of partially laminating a polymer lipid membrane (b) on a surface of a substrate (a), followed by lamination of a fluid lipid membrane (c) on the surface of the substrate (a), on which the polymer lipid membrane (b) is free from being laminated, together with a membrane protein (d) in a nanodisc containing an amphipathic peptide, wherein the membrane protein (d) is reconstituted under a state of retaining lateral diffusivity.

2. The method of producing an artificial biological membrane according to claim 1, wherein the step of partially laminating the polymer lipid membrane (b) on the surface of the substrate (a) is performed by: causing a lipid membrane of a polymerizable lipid (monomer) to adsorb to the surface of the substrate (a); and forming the polymer lipid membrane (b) through photopolymerization.

3. The method of producing an artificial biological membrane according to claim 1, wherein the method comprises the following steps (1) to (4):
(1) a step of causing a lipid membrane of a polymerizable lipid (monomer) to adsorb to the surface of the substrate (a);
(2) a step of protecting part of the adsorbing lipid membrane from photoirradiation, followed by application of light to the polymerizable lipid (monomer) that is free from being protected from the photoirradiation to polymerize the monomer, to thereby subject the polymer lipid membrane (b) to pattern formation on the surface of the substrate (a);
(3) a step of removing the lipid membrane, which has been protected from a photopolymerization reaction in the step (2), from the surface of the substrate (a) with a detergent or an organic solvent; and
(4) a step of laminating the fluid lipid membrane (c) on a site of the surface of the substrate (a), from which the lipid membrane has been removed in the step (3), together with the membrane protein (d).

4. An artificial biological membrane, comprising:
a substrate (a); and
a lipid membrane laminated on a surface of the substrate (a),
wherein the lipid membrane to be laminated includes a polymer lipid membrane (b) and a fluid lipid membrane (c) containing a membrane protein (d), and
wherein the membrane protein (d) is in a state of retaining lateral diffusivity.

5. The artificial biological membrane according to claim 4,
wherein the polymer lipid membrane (b) is partially laminated on the surface of the substrate (a), and
wherein the fluid lipid membrane (c) containing the membrane protein (d) is laminated on the surface of the substrate (a) on which the polymer lipid membrane (b) is free from being laminated.

6. The artificial biological membrane according to claim 4, wherein the polymer lipid membrane (b) is a photopolymerizable lipid membrane.

7. The artificial biological membrane according to claim 4,
wherein the polymer lipid membrane (b) is subjected to pattern formation to be laminated on the surface of the substrate (a), and
wherein the fluid lipid membrane (c) containing the membrane protein (d) is laminated on the surface of the substrate (a) on which the polymer lipid membrane (b) is free from being laminated.

8. An artificial biological membrane, comprising:
a substrate (a); and
a lipid membrane laminated on a surface of the substrate (a),
wherein the artificial biological membrane is produced by the production method of claim 1, and
wherein a membrane protein (d) is reconstituted under a state of retaining lateral diffusivity.

9. The artificial biological membrane according to claim 4, wherein the membrane protein (d) is one or two or more kinds of membrane proteins.

10. The artificial biological membrane according to claim 9, wherein at least one kind of the membrane protein (d) is a protein classified into a G protein-coupled receptor (GPCR).

11. A method of analyzing a function of a membrane protein (d), the method comprising using the artificial biological membrane of claim 4.

12. A method of evaluating a substance targeting a membrane protein (d), the method comprising using the artificial biological membrane of claim 4.

13. A device for an inspection concerning a membrane protein (d), the device comprising the artificial biological membrane of claim 4.

14. A kit for an inspection concerning a membrane protein (d), the kit comprising at least the device for an inspection of claim 13, and further comprising a reagent and/or an instrument required for the inspection of the membrane protein (d).
